(19) United States
(12) Patent Application Publication
(10) Pub. No.: US 2010/0114007 A1
(43) Pub. Date: May 6, 2010

(54) DERMATOLOGICAL TREATMENT APPARATUS

(75) Inventors: Dietmar Fischer, Erlangen (DE); Daniel Haberer, Forchheim (DE); Sebastian Kaukel, Hemhofen (DE); Henry Parusel, Quellendorf (DE); Arnold Pribbernow, Simmelsdorf (DE); Udo Schmidt, Heroldsberg (DE); Stefan Schulze, Nurnberg (DE); Martin Winkler, Hohenstein-Ernsttal (DE)

Correspondence Address: HAYNES AND BOONE, LLP;IP Section, 2323 Victory Avenue, Suite 700, Dallas, TX, 75219, US

(73) Assignee: OUANTEL DERMA GMBh, Erlangen, DE (US)

(21) Appl. No.: 12/444,831

(22) PCT Filed: Oct. 9, 2007
(86) PCT No.: PCT/EP07/08769

§ 371 Date: Dec. 18, 2009

(30) Foreign Application Priority Data
Oct. 10, 2006 (EP) 06021252.9

(51) Int. Cl. A61B 18/20; A61B 18/18

(52) U.S. Cl. 604/20; 606/9

(57) ABSTRACT

An apparatus for dermatological treatments has a base equipment 10 to which differing application heads 14 can be connected, selectively via flexible conduits 12, for the purpose of applying radiation, in particular of an LED and of a laser or of a high-energy flashbulb.

The invention relates to an apparatus for dermatological treatments, with which skin can be irradiated with electromagnetic radiation.

Such an apparatus is known from U.S. Pat. No. 6,273,884 B1, for example. Therein an application head is guided by the operating surgeon over the skin to be treated. WO 2004/004831 A1 also describes a similar system.

In the known systems for dermatological treatments of the skin with electromagnetic radiation a so-called base equipment (base station) is provided, and the physician can connect a so-called application head to the base equipment via a flexible (bendable) conduit. A radiation-source may be arranged either directly in the application head or in the base equipment. In the latter case the flexible conduit then contains a light guide, in order to transmit the radiation generated in the base equipment to the application head.

The invention sets itself the object to provide an apparatus for dermatological treatments that, with relatively little effort in terms of apparatus, enables an effective treatment of the skin with radiation.

For this purpose the invention provides an apparatus for the dermatological treatment, with a base equipment to which at least one application head for applying radiation onto skin is connected via conduits, the application head comprises a device for applying a pharmaceutical substance or health-care substance onto the skin.

Preferred configurations of this apparatus are described in dependent Claims 2 to 5.

The invention also provides an apparatus for dermatological treatments, with a base equipment to which via flexible conduits selectively at least one first application head for applying radiation of an LED onto skin and/or at least one second application head for applying radiation of a laser or of a high-energy flashbulb onto skin is/are capable of being connected.

The invention accordingly enables, with a single base equipment, the optional connection of a first application head for irradiating skin with radiation emitted from one or more LEDs and/or the connection of a second application head via another flexible conduit, so that radiation of either a laser or of a high-energy flashbulb can be applied onto skin.

For the treatment of the skin with differing electromagnetic radiation, a physician or hospital therefore does not necessarily need to acquire and keep a specific base equipment for each type of radiation, but, on the contrary, a single base equipment serves for the supply and control of differing application heads with, in each instance, differing types of electromagnetic radiation.

Hence the physician is enabled, with a single relatively uncomplex equipment system, to carry out differing medical treatments. Corresponding remarks apply in respect of a beautician.

The wide range of application of dermatological treatments that is thereby made possible includes, for example, epilation (hair-removal) or PDT (photodynamic therapy). These treatments require differing types of radiation (wavelength, energy, time, add-on devices etc.). With the system according to the invention, operation is also simplified, since the same base equipment with the same operating elements is employed for all applications. A further advantage of the system according to the invention lies in the fact that a user is able to retrofit optional application heads to a given base equipment.

The base equipment according to the invention also enables light-sources or laser-sources to be connected via a fibre coupling, which can then be employed for other treatments in the body (e.g. ENT, surgery, urology, cardiology).

According to one configuration of the invention, the LED and/or the laser is/are arranged in each instance in an assigned application head.

Another configuration of the invention provides that the LED and/or the laser and/or the flashbulb is/are arranged in the aforementioned base equipment, so that the radiation is capable of being transmitted by light guides to the respectively provided application heads.

Also a plurality of the stated radiation-sources of the same type may arranged in the base equipment or in the application head, respectively.

An LED emitting in a narrow band is preferably employed.

Another preferred configuration provides that the LED emits with intensities that are designed to achieve photobiological effects in the skin.

Whenever the term ‘LED’ is mentioned here, this also encompasses OLEDs (organic light-emitting diodes).

Another preferred configuration of the invention provides that the LED or the plurality of LEDs emit(s) in the UVA region. LEDs may also be provided that emit in the UVB region.

Another variant of the invention provides that two or more LEDs that emit with differing wavelengths are arranged in one application head, in particular for homogeneous illumination of an area of skin to be treated.

Application heads may also be provided that enable differing LEDs to be selectively employed.

The laser that is employed in accordance with the invention additionally the LEDs may, in particular, be a solid-state laser, a diode laser, and, furthermore in particular, a pulsed IR laser, each with intensities for achieving photothermal effects in the skin. Thereby the solid-state laser may preferably be a CW laser.

Another preferred configuration of the invention provides an application head, with devices for applying a liquid or cream onto the skin. Such an application head is also an independent subject of this application and is to be communicated as being disclosed and claimable independently of the other features.

Another configuration of the apparatus provides that an application head comprises means for projecting radiation into a predetermined pattern, in particular into a narrow bar or line, and for scanning (moving) the pattern over the skin. In this process the application head remains stationary, and only the radiation projected onto the skin in the pattern moves over the skin. This configuration of the invention is also to be regarded here as being disclosed and claimable on its own, independently of the other features of the invention.

Another preferred configuration of the invention provides that the base equipment and the assigned differing application heads each comprise means with which the base equipment is able to ‘recognise’ a connected application head and thereupon automatically sets the parameters (power supply, where appropriate, radiation control etc.) appropriately, so that the user at the base equipment does not have to perform any special adjustments depending on the type of the application head. For example, in each application head a transmitter may be provided which, when the application head is supplied with current, transmits to the base equipment via the aforementioned flexible conduit a coded signal which the base equipment is able to read, in order to recognize the type of the application head, and thereupon selects the required and assigned control parameters for this application head and indicates to the user on a display panel what is provided in terms of control and what he/she still has to adjust where appropriate.

Embodiments of the invention will be explained in more detail below based on the drawings.

Shown are in:

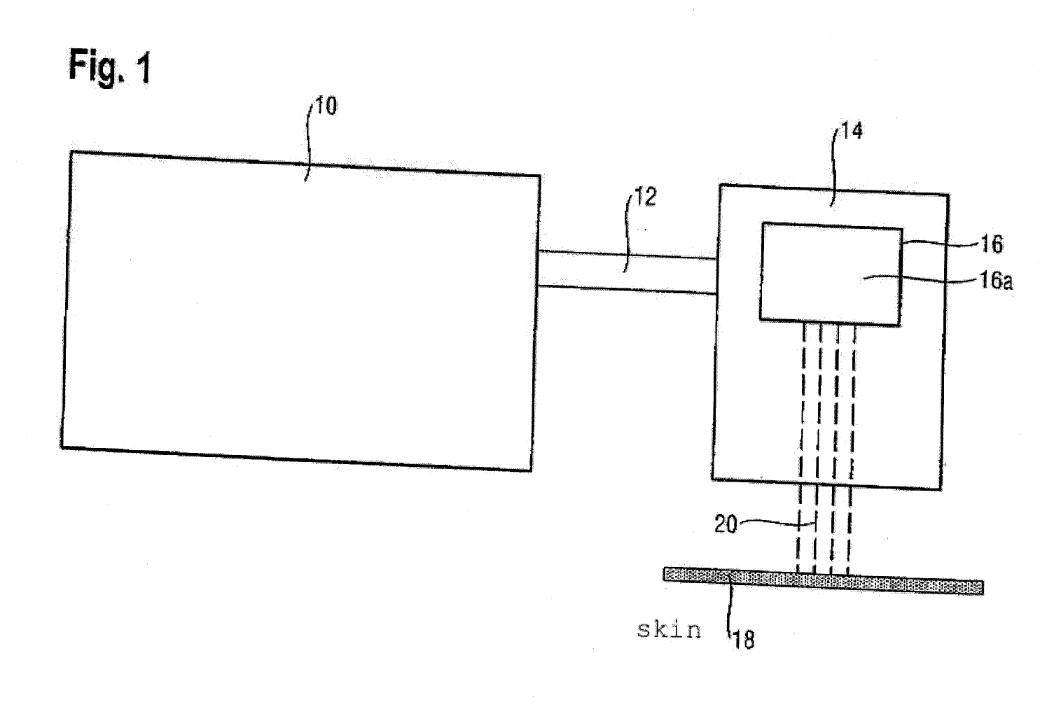

FIG. 1 a first embodiment of an apparatus for dermatological treatments, with a base equipment and an application head;

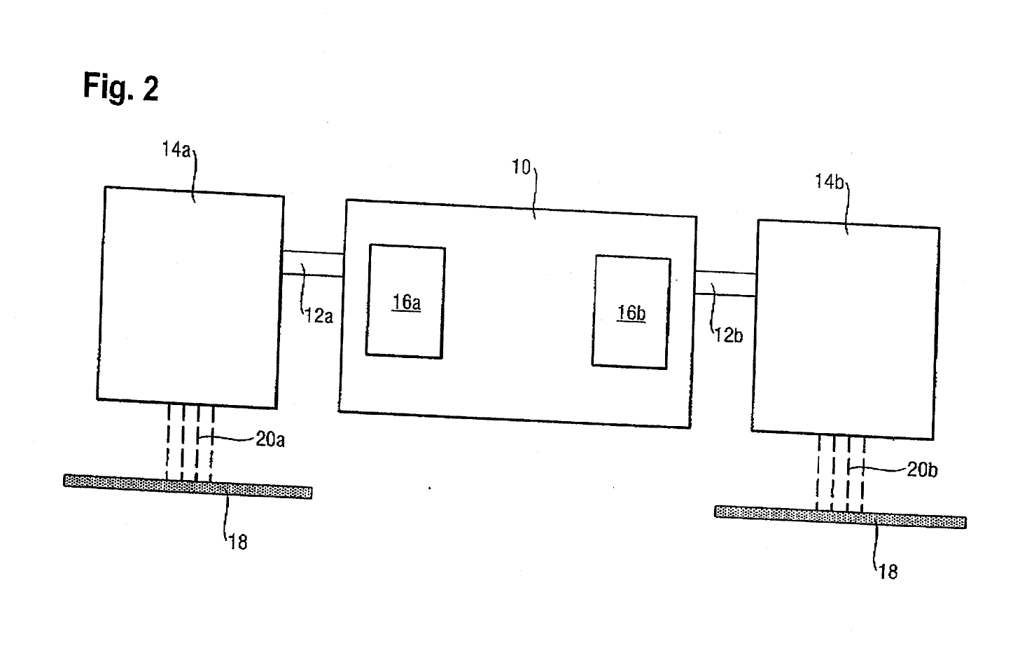

FIG. 2 a second embodiment of an apparatus for dermatological treatments, with a single base equipment to which two different application heads are connected;

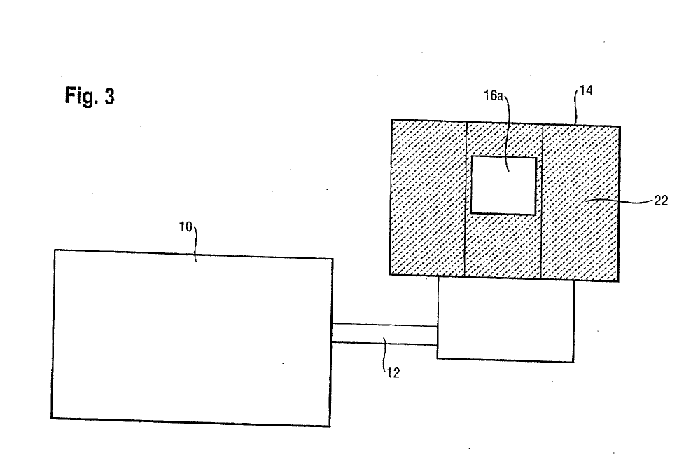

FIG. 3 a further embodiment of an apparatus for dermatological treatments, with a base station and with an application head which exhibits a so-called face mask;

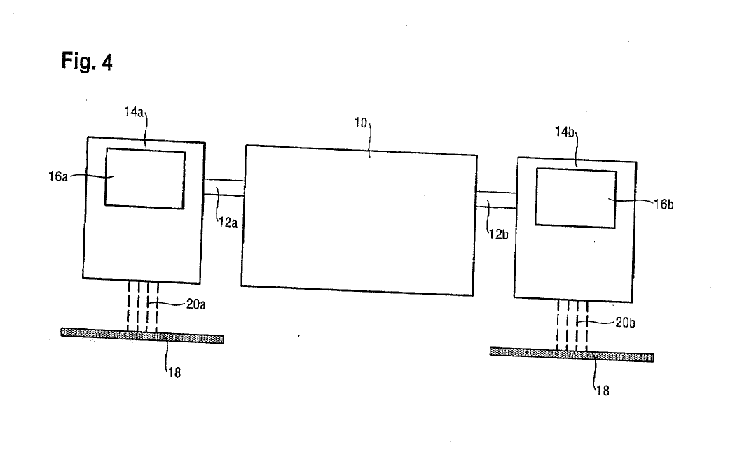

FIG. 4 a modification of the apparatus according to FIG. 2, wherein the light-sources are arranged in the application heads;

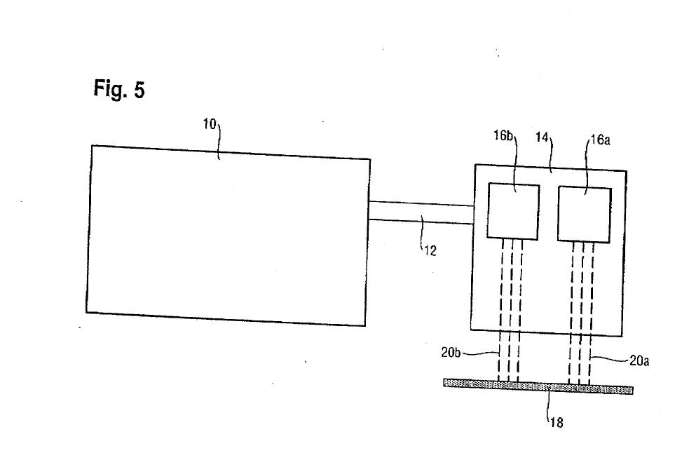

FIG. 5 a modified embodiment of an apparatus for dermatological treatments, with an application head in which two differing light-sources are arranged;

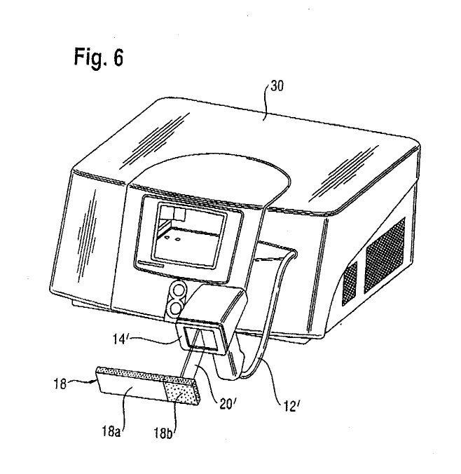

FIG. 6 a further embodiment of an apparatus for dermatological treatments, with an application head and with optical means arranged therein for projecting radiation into a fine bar and also with means for oscillating movement of the projected radiation bar over skin;

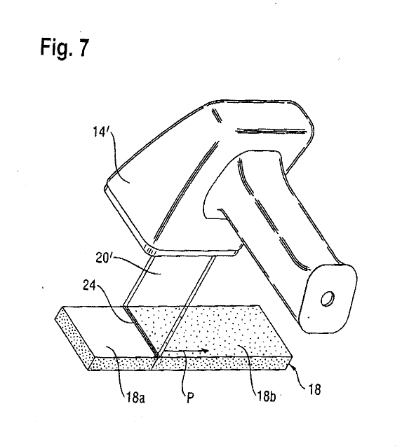

FIG. 7 the apparatus according to FIG. 6 in a different representation without the base equipment;

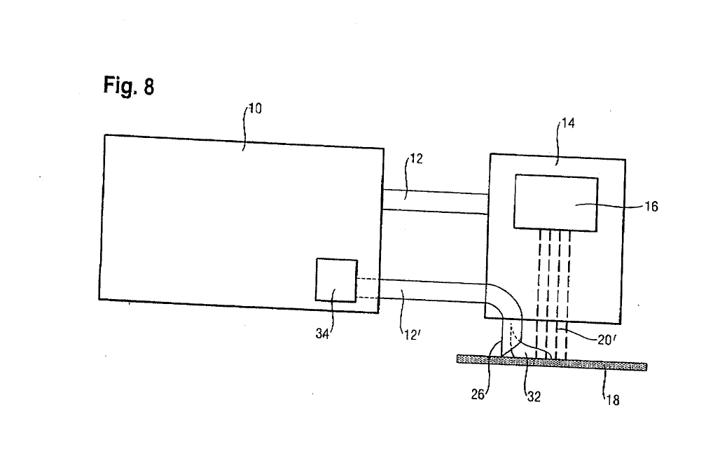

FIG. 8 a further embodiment of an apparatus for dermatological treatments, with devices for applying cream or liquid onto the skin;

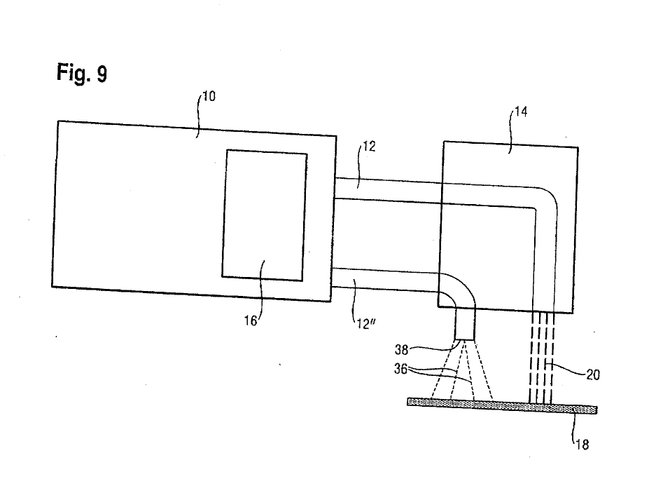

FIG. 9 a further embodiment of an apparatus for dermatological treatments, with an apparatus for spraying liquid agents or cream-like agents onto skin;

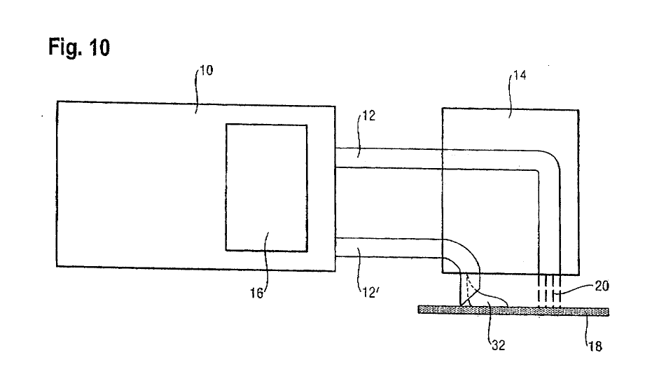

FIG. 10 a modification of the apparatus according to FIG. 8, wherein the light-source is arranged in the base equipment;

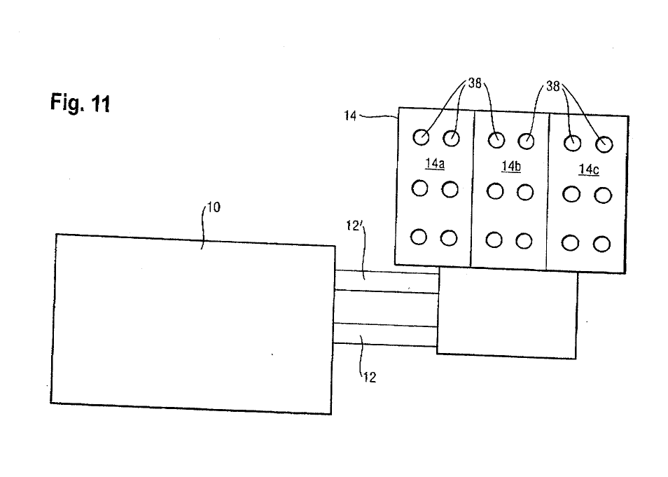

FIG. 11 a further variant of an apparatus for dermatological treatments, wherein an application head exhibits a face mask with spray holes for applying liquid or cream onto the skin; and

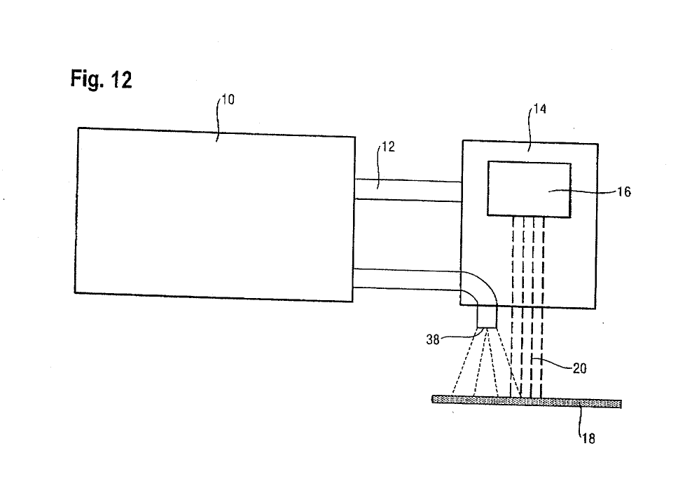

FIG. 12 a modification of the embodiment according to FIG. 9, wherein the light-source is arranged in the application head.

The Figures show schematically the individual components of systems for dermatological treatments of the skin with radiation. Components that correspond to one another or that are functionally similar are provided with the same reference numerals, where appropriate supplemented by letters or other distinguishing features.

A base equipment 10 is connected to an application head 14 via a flexible conduit 12. In the application head 14 there is a light-source 16, here for example an LED 16a. The radiation 20 emitted from the light-source 16 is directed onto skin 18.

The base equipment 10 includes the power supply also for the application head 14, electronic controllers for the application head 14 and the base equipment itself, and operating elements for the user for the purpose of setting parameters. Consequently the flexible conduit 12 includes, in particular, electrical conductors, where appropriate (for example if, as in the case of exemplary embodiments described further below, the light-source is arranged in the base equipment), light guides, and, where appropriate, also coolant conduits etc.

In the embodiment according to FIG. 1 the light-source 16 is accommodated in the application head 14. In this case the light-source 16 may be, in particular, an LED, a laser, or a high-energy flashbulb. Three or more differing application heads 14 may respectively be provided appropriately, which are selectively connectable to the base equipment 10 and which receive the required supply signals and control signals from the base equipment. Each of the various application heads 14 is accordingly connected to the base equipment via a connection-point provided therefore on the base equipment 10 via the respectively assigned connecting conduit 12.

FIG. 2 shows a modified embodiment with a base equipment 10 in which an LED 16a is provided as a light-source and a laser 16b is provided as a further light-source. Via respectively assigned flexible conduits 12a and 12b the emitted radiations of the LED 16a and of the laser 16b are respectively transmitted to the applicator. Hence it is possible to apply differing radiations 20a, 20b onto the skin 18 simultaneously or successively.

FIG. 3 shows a modification of the embodiment according to FIG. 1, wherein a so-called face mask is formed on the application head 14. With a face mask, larger body-parts can be irradiated accurately. Thereby, the term ‘face mask’ not only encompasses the actual face but may also correspond to other body-parts that are to be treated. The mask is accordingly at least approximately modelled on the shape of the body-part to be treated and thereby enables an accurate and reproducible positioning of the application head. In the face mask several light-sources—in particular, LEDs or lamps—may be arranged in distributed manner. Otherwise the embodiment according to FIG. 3 corresponds to the variants according to FIGS. 1 and 2.

FIG. 4 shows a modification of the exemplary embodiment according to FIG. 2, in that the light-sources have been relocated from the base equipment 10 into the application heads 14a, 14b. In the application head 14a one or more LEDs 16a are arranged, and in the application head 14b one or more lasers 16b are arranged. Otherwise the configurations correspond to the exemplary embodiments described above.

FIG. 5 shows a variant of an apparatus for dermatological treatments, wherein in one application head 14 two different light-sources are arranged, namely, on the one hand, an LED 16a (or several LEDs 16a) and, on the other hand, a laser 16b (or several lasers 16b). The two light-sources may selectively be activated individually or jointly.

In the exemplary embodiments described based on FIGS. 1 to 5 the following light-sources may be employed in particular:

As a laser, an IR diode laser which emits at 808 nm or 980 nm. Such radiation is well suited for epilation, for light skin or dark skin, respectively, according to the chosen wavelength.

For the LEDs, UV LEDs may be chosen that emit at 308 nm or 370 nm, for example. This radiation is well suited for the treatment of autoimmune infirmities.

Also employable are blue-emitting LEDs for treating acne or even for PDT. Yellow-emitting LEDs (for example, 590 nm) are particularly suited for the treatment of acne and for skin rejuvenation. Red-emitting LEDs (640 nm) are particularly suited for healing wounds and also for PDT.

The application heads described above (and below) may also be provided with means in order to determine skin parameters, for example the temperature, the tanning of the skin, the melanin content, the thickness of the epidermis etc. Means may also be provided for generating images of the treatment surface.

FIG. 6 shows a special configuration of an application head 14′ which is provided with optical and electro-optical means in order to project the electromagnetic radiation 20′ emitted by it on the skin 18 in a narrow bar. The bar may have, for example, transverse dimensions from 0.5 mm to 5 mm, in particular 0.5 mm to 2 mm. Its longitudinal dimension amounts to a multiple of the transverse dimension, for example 2 times to 20 times the transverse dimension.

FIG. 7 shows the application head 14′ from FIG. 1 from a different perspective, viewed in the direction towards the surface of the skin 18 to be irradiated. As is shown, the optical means in the application head 14′ generate a radiation 20′ which projects a bar-like pattern 24 on the skin 18. The bar 24 is scanned in the direction of the arrow P. The bar 24 accordingly retains its shape and dimension during the scanning and is moved over the skin 18 in the direction of the arrow. The already treated portion of the skin is denoted in FIG. 7 by 18a, and the portion of the skin still to be treated is denoted by 18b.

This arrangement makes it possible to operate with relatively small spot-sizes without reducing a depth-effect that is required for follicle heating. By virtue of the continuous irradiation, a sufficient heating and an accumulation of the heat in the skin occur. Relatively weaker CW lasers can be employed and optimally utilised. By reason of the focusing in a bar, the requisite CW power also falls. Diode lasers and solid-state lasers may be employed. Thereby, the variants of the arrangement of the light-sources in the system are possible that have been described on the basis of FIGS. 1 to 5.

By reason of the continuous traversing with the scanner of the region of skin to be epilated, relatively weak diode lasers (for example, with a power of 300 W) may, for example, be employed for rapid epilation. By virtue of the fact that the radiation is not switched on and off, a larger percentage (more than 50%) of the CW-laser power that is generally available can be employed for the epilation, resulting altogether in a higher speed of treatment. The stated small spot dimensions (length times width of the bar) make small, inexpensive and simple-to-cool laser-diode modules possible, which are also to be supplied with relatively low power, without adversely influencing the user-friendliness and also the depth of penetration into the skin. Small, manageable and easily transportable instruments can be produced.

As distinct from the state of the art according to U.S. Pat. No. 6,273,884, FIG. 12A therein, in the embodiment of the apparatus that has been explained based on the FIGS. 6 and 7 the scanning occurs perpendicular to the longitudinal direction of the bar (direction of the arrow P).

In the embodiment according to FIGS. 6 and 7, laser light with wavelengths in the range between 650 nm and 1200 nm is preferably employed. If the bar 24 has, for example, dimensions of 0.3 cm×1 cm, the geometrical parameters of the scanning can be set in such a way that, for example, a skin-treatment area of 5 cm×1 cm is traversed (scanned), whereby the dimension of 1 cm corresponds to that of the longitudinal dimension of the bar 24. The scanning is controlled in such a way that the treatment area is swept continuously by the, in relation thereto, substantially smaller bar 24, i.e. the incident light radiation is not switched on and off during the scanning. In practice, this means that working proceeds with very long pulse durations, for example pulse durations within the range from more than 200 ms to 2 s, and light is continuously beamed onto the skin during these pulse durations. The continuous scanning is controlled in such a way that a point on the tissue is irradiated with light only for about 5 ms to 50 ms, in order to avoid burns. The other relevant parameters—such as pulse duration, fluence and scan region—can be adjusted by the user, taking account of the stated boundary conditions.

As light-sources, lasers—in particular CW-radiating lasers such as diode lasers, for example—are considered, but also LEDs, including OLEDs. Also suitable are lamps, the light of which can be focused in the described manner.

With regard to the arrangement of the light-sources, the variants described on the basis of FIGS. 1 to 5 are possible. Scanners and the drives thereof are well-known as such in the state of the art; they operate with movable mirrors and lenses. Alternatively, a lightweight light-source arranged in the application head 14′ could also be moved, in order to achieve the scan procedure.

With the power and time parameters described above, hair follicles in deeper dermal layers (approx. 500 μm to 4 mm) can be reached and heated.

Instead of a strict CW operation, a quasi CW operation is also sufficient, i.e. short pulse durations longer than 1 ms with comparatively short pulse pauses, which are shorter than the pulses themselves, may be employed, or very short pulses shorter than 0.5 ms may also be employed, with pulse pauses within the same time-range.

The apparatuses described above preferably exhibit means with which the amounts of energy that are used for the cooling and/or light-generation are capable of being changed temporally in such a way that the average and/or peak light outputs at predetermined current input power (this is the so-called connected load) are maximised.

FIG. 8 shows a configuration with a base equipment 10 and with an applicator 14 as well as a flexible conduit 12′ for transferring a cream or a liquid from a reservoir 34 to the applicator 14. From the applicator 14 there protrudes a drop-dispenser 26 which applies a drop 32 onto the skin 18. The application head accordingly enables the combination of a phototherapy with pharmaceutical substances or health-care substances to be applied topically. The apparatus makes it possible to apply substances in liquid form—in particular creams, ointments or sprays—or in gaseous form onto the skin shortly before, during or shortly after a phototherapy, in order to promote the treatment or to reduce side-effects.

The variants of the apparatus for dermatological treatments described in FIGS. 8 to 12 make it possible to apply pharmaceutically active substances or health-care substances substantially in parallel, temporally and spatially, with the phototherapy.

For the operating surgeon this combined treatment is possible in straightforward manner with the systems according to FIGS. 8 to 12.

In a modification of the exemplary embodiments that have been described, cooling air can also be applied onto the skin via a conduit analogous to the flexible conduit 12′.

FIG. 9 shows a variant with a spray device. Spray 36 is applied onto the skin 18 from a nozzle 38. With respect to the substances, what was said with reference to FIG. 8 applies.

FIG. 10 shows a modification of the apparatus according to FIG. 8, with a light-source 16 in the base equipment 10.

FIG. 11 shows a configuration of an application head 14 with a so-called face mask (see above). Pharmaceutical substances and/or health-care substances can be applied in temporally and quantitatively defined controlled manner onto the skin via a plurality of nozzles 38 which are distributed over a large area of the face mask.

FIG. 12 shows a modification of the embodiment according to FIG. 8, with a spray apparatus (nozzle 38) instead of the dispenser applying a liquid or a drop directly onto the skin.

In the embodiments according to FIGS. 8 to 12 the reservoir for the health-care substance or pharmaceutically active substance may, in modification of the foregoing description, also be arranged in the application head or even in a face mask itself. In these cases the conduit 12′ can be dispensed with.

1.-12. (canceled)

13. Apparatus for dermatological treatments of the skin, comprising a base equipment to which at least one first application head for applying radiation of an electromagnetic radiation-source onto the skin can be connected via flexible conduits,
characterised in that the first application head comprises optical means for projecting radiation in a linear pattern and scanning means for moving the linear pattern over the skin in relation to the application head perpendicular to the longitudinal axis thereof.

14. Apparatus according to claim 13, characterised in that a second application head for applying radiation onto the skin is connectable to the apparatus via further flexible conduit, one of the first or second application heads applying radiation of an LED onto skin and the other of the application heads applying radiation of a laser or of a high-energy flashbulb onto the skin.

15. Apparatus according to claim 13, characterised in that the pattern is rectangular, with a ratio of long side to short side of more than 2.

16. Apparatus according to claim 14, characterised in that at least one of the LED or the laser is disposed in the first application head.

17. Apparatus according to claim 14, characterised in that at least one of the LED, the laser or the flashbulb is disposed in the base equipment.

18. Apparatus according to claim 13, wherein the electromagnetic radiation-source is an LED that emits in a narrow band with a spectral width of less than 50 nm.

19. Apparatus according to claim 13 wherein the electromagnetic radiation-source is an LED that emits with intensities that are designed for achieving photobiological or photochemical effects in the skin.

20. Apparatus according to claim 13 wherein the electromagnetic radiation-source is an LED that emits in the UVA region.

21. Apparatus according to claim 13 wherein the electromagnetic radiation-source is an LED that emits in the UVB region.

22. Apparatus according to claim 1, having a first application head that includes two or more LEDs having differing wavelengths providing homogeneous illumination of an area of skin.

23. Apparatus according to claim 22, further including a switch to independently control the two or more LED's such that differing LEDs are optionally capable of being employed in the application head.

24. Apparatus according to claim 14, wherein the laser is a solid-state IR laser having intensities sufficient for achieving photothermal effects in the skin.

25. Apparatus according to claim 24, characterised in that the solid-state IR laser is a CW diode laser.

26. Apparatus according to claim 13, wherein said first application head includes a device for applying a liquid or cream onto the skin.

27. Apparatus according to claim 26, wherein the device sprays the liquid or cream onto the skin.

28. Apparatus according to claim 26, wherein the device drips liquid or cream onto the skin.

29. Apparatus according to claim 13, wherein at least one of the application head and the flexible conduit include an identifier and the base equipment comprises a reader operable to recognize the identifier.

30. An apparatus for dermatological treatments of the skin for use with a base station having at least a power supply, the apparatus comprising
an application head for applying therapeutic radiation of an electromagnetic radiation-source onto the skin to achieve photobiological or photochemical effects in the skin;
a flexible conduit connected between said base station and said application head,
wherein the application head includes a guide configured to project the radiation in a pattern having a longitudinal axis and a scanner controllable to move the pattern over the skin in relation to the application head generally perpendicular to the longitudinal axis thereof.

31. The apparatus of claim 30, wherein the electromagnetic radiation-source is included within said application head.

32. The apparatus of claim 31, wherein the application head includes at least two different types of radiation sources and a switch to independently control the radiation sources.
